(19) United States
(12) Patent Application Publication
(10) Pub. No.: US 2007/0213788 A1
(43) Pub. Date: Sep. 13, 2007

(54) Electrical stimulation of the inner ear in patients with unilateral hearing loss

(76) Inventors: Mary Joe Osberger, Nanuet, NY (US); Albert A. Maltan, Sistrans (AT)

Correspondence Address: ADVANCED BIONICS CORPORATION, 25129 RYE CANYON ROAD, VALENCIA, CA, 91355, US

(21) Appl. No.: 10/941,207

(22) Filed: Sep. 15, 2004

Related U.S. Application Data
(60) Provisional application No. 60/504,064, filed on Sep. 19, 2003.

(51) Int. Cl. A61N 1/00

(52) U.S. Cl. 607/57

(57) ABSTRACT

For a patient having unilateral hearing loss, an extra-cochlear electrode is placed within the middle ear of the deaf ear, and is connected to a microstimulator that is implanted under the skin or recessed in the temporal bone or some other location in the skull near the deaf ear. In one preferred embodiment, the extra-cochlear electrode is a mesh ball electrode that is placed in the round window niche, on the promontory, or in some other extra-cochlear location, of the deaf ear. Whenever a trigger signal generated by a microphone is received by the microstimulator, the microstimulator generates a stimulus pulse that is applied to the mesh ball electrode. The electrical signal applied through the mesh ball electrode provides temporal information to supplement the sound signal that is received by the other, normally-functioning ear of the patient.

The present application claims the benefit of U.S. Provisional Patent Application Ser. No. 60/504,064, filed 19 Sep. 2003, which application is incorporated herein by reference in its entirety.

### BACKGROUND OF THE INVENTION

The present invention relates to the use of electrical stimulation of the inner ear in patients suffering from unilateral hearing loss.

When hearing is impaired in only one ear, it is called a unilateral hearing loss. The following are some reasons a person may have a unilateral hearing loss: ear wax that is completely occluding the ear canal, middle ear fluid or other types of ear infections, cranial radiation, a tumor pressing on or near the auditory nerve, surgery or swelling close to the auditory nerve, a hole in the ear drum, and unilateral hearing loss at birth for unknown reasons. An individual experiencing unilateral hearing loss will have problems when a soft-spoken person speaks to them from the poorer side. They may also have problems localizing the direction of sound (sound localization requires that both ears have approximately identical hearing). In noisy backgrounds the better ear will behave like a funnel and all sound, including speech and background noise, will enter the funnel and get mixed together with little ability to distinguish the perceived noise.

Cochlear implantation of deaf or hearing-impaired individuals is currently recommended only in the case of bilateral deafness. People with unilateral hearing loss suffer many significant disadvantages in everyday listening situations, especially in the presence of background noise. Yet, placement of a commercial multi-channel cochlear implant in the unilaterally deaf ear is deemed not financially viable due to the high cost of the procedure.

What is needed is a cost-effective electrical stimulation system whereby people with unilateral hearing loss can benefit from electrical stimulation of the inner ear.

### SUMMARY OF THE INVENTION

The present invention addresses the above and other needs in patients suffering from unilateral hearing loss by placing an extra-cochlear electrode within the middle ear of the deaf ear, and connecting such electrode to a microstimulator that is implanted under the skin or recessed in the temporal bone or some other location in the skull near the deaf ear. In one preferred embodiment, the extra-cochlear electrode comprises a mesh ball electrode that is placed in the round window niche, on the promontory, or in some other extra-cochlear location, of the deaf ear.

The mesh ball electrode is connected to the microstimulator via a thin wire that is routed, through a standard middle ear surgical procedure that can be performed under local anesthesia, behind the skin and along the bone of the ear canal.

In one preferred embodiment, the microstimulator contains its own power supply, e.g., a rechargeable power supply, or may receive operating power from an external power source through close-field RF coupling.

A microphone, or other external signal-gathering and signal processing device, is coupled to the microstimulator, and includes the ability to generate a trigger signal (or other control signal) whenever the acoustic signals sensed through the microphone meet specified criteria. Whenever the trigger signal or other control signal is received by the microstimulator, the microstimulator generates a stimulus pulse that is applied to the mesh ball electrode.

The electrical signal applied through the mesh ball electrode provides temporal information to supplement the sound signal that is received by the other, normally-functioning ear of the patient.

### BRIEF DESCRIPTION OF THE DRAWINGS

The above and other aspects, features and advantages of the present invention will be more apparent from the following more particular description thereof, presented in conjunction with the following drawings wherein:

FIG. 1 is a side cross-sectional view of a mesh, ball electrode that may be used with the present invention;

FIG. 2 is a sectional view of the middle ear, and illustrates a representative placement of the mesh, ball electrode;

FIG. 3 is a side view of relevant portions of the middle-/inner-ear interface, and illustrates a preferred manner of placing the mesh, ball electrode in the niche or recess in front of the round window; and

FIG. 4 is a sectional view of relevant portions of the middle and outer ear, and illustrates a preferred placement of an implantable neurostimulator, such as the BION stimulator, that is electrically connected to the mesh, ball electrode within the middle ear in accordance with the present invention.

Corresponding reference characters indicate corresponding components throughout the several views of the drawings.

### DETAILED DESCRIPTION OF THE INVENTION

The following description is of the best mode presently contemplated for carrying out the invention. This description is not to be taken in a limiting sense, but is made merely for the purpose of describing the general principles of the invention. The scope of the invention should be determined with reference to the claims.

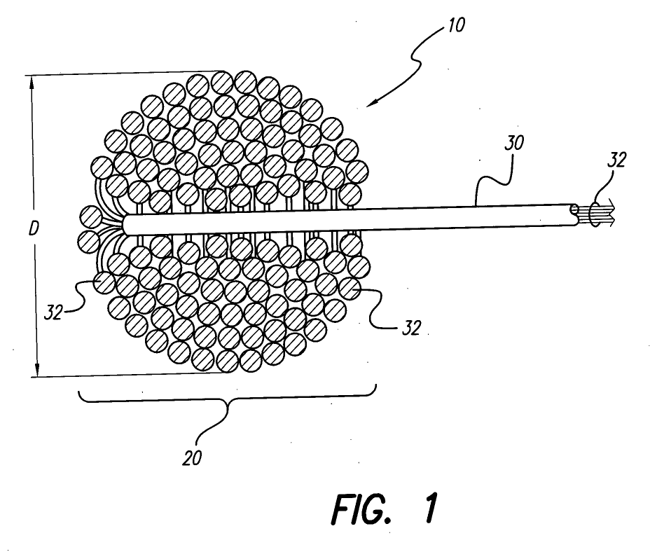

Turning first to FIG. 1, a cross-sectional side view of a mesh, ball electrode is shown. A mesh, ball electrode 10 is made by wrapping the wires of a cable 30 around a suitable mandrel (not shown) to form a ball-shaped head 20 having a diameter “D” from 1.5 to 2.5 mm. The cable 30 is preferably made from an insulated multi-strand cable, having multiple wires or strands 32. In one embodiment, the cable 30 may be made from Teflon-insulated 9- or 11-strand Pt/Ir wires 32. The length of the wires 32 may be about 200 mm, sixty (60) mm of which forms the cable 30, forty (40) mm of which extends out from the cable, e.g., so that the wires can be connected to a suitable neurostimulator, and sixty-to-one hundred (60-100) mm of which are used to form the head 20 of the ball electrode 10. Each lead wire 32 is, at a proximal end, welded to platinum pins (not shown) on a neurostimulator, or to a connector that attaches to a neurostimulator, or to a BION-type stimulator, or otherwise electrically connected to a suitable stimulation device.

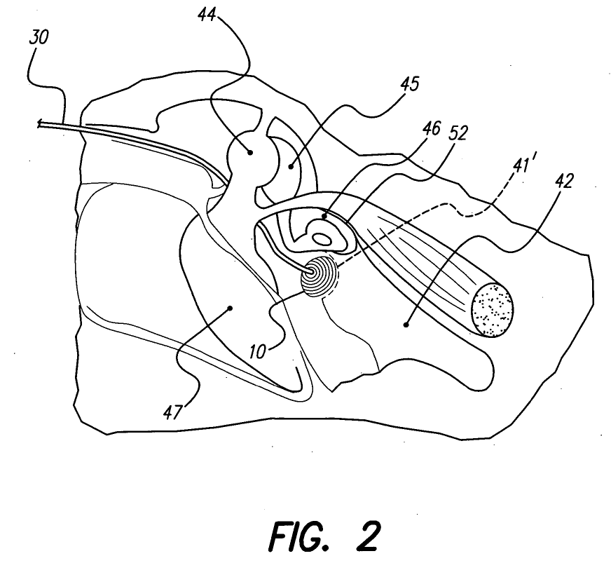

To form the ball-shaped head 20 of the electrode, a sixty-to-one hundred (60-100) mm length of insulated wire 32 is stripped and annealed at a temperature of 1000-1200 C., after which it is allowed to cool at room temperature. Then, the wire is wrapped using a mandrel (not shown), as generally described in FIGS. 2B-2F of U.S. Pat. No. 4,809,712, incorporated herein by reference.

The mandrel has a diameter of about 0.45 mm and a tip having a length of between about 1.5-2.5 mm. A notch having a width of about 0.15 mm is also located at the tip. The notch is placed around the end of the remaining insulation of the cable 30, while the wires or strands 32 are wrapped around the mandrel twenty-five to forty times (depending upon the diameter of the ball that is desired) to form the ball electrode 10 with unfixed turns and an outer diameter “D” of 1.5-2.5 mm. Once the ball shaped head 20 is formed, the mandrel is pulled gently away from ball electrode 10, leaving the ball shaped head 20 intact. FIG. 1 illustrates a cross-section of the ball electrode 10. Note that the ball electrode is porous in the sense that the winding process leaves spaces between adjacent turns.

FIG. 2 illustrates the mesh, ball electrode 10 used with the present invention positioned in one preferred location in front of the round window 42. As illustrated in FIG. 2, the cable 30 may re routed through the middle ear, past the malleus 44, incus 45, and stapes 46, without significantly interfering with their normal operation, thereby preserving residual hearing. One advantage of the present invention is that such cable 30 may be routed through the middle ear using standard middle ear surgical procedures performed under a local anesthesia, behind the skin and along the bone of the ear canal, to the microstimulator, or other neurostimulator, which is placed under the skin or recessed in the temporal bone or other suitable location in the skull.

An outline of the normal cavity, niche, or recess, that is located on the middle ear side of the round window 42 is depicted by the dotted line 41′. Applicants have discovered that by placing the mesh ball electrode 10 within this cavity, or recess, or in another suitable extra-cochlear location of the non-functioning (or deaf) ear, and by then applying an electrical stimulus through this electrode, sufficient temporal information is provided to the middle-ear/inner-ear of the non-functioning ear to supplement the sound signal that is received through the other, normally-functioning ear. Advantageously, placement of this extra-cochlear electrode may be accomplished under local anesthesia, thereby significantly reducing the cost and trauma associated with cochlear implant surgery.

Thus, it is seen that the stimulation provided through the ball electrode 10 assists with the normal hearing processes. That is, by providing electrical stimulation through the mesh ball electrode 10 to the middle-ear side of the round window 42, or to another suitable extra-cochlear location of the deaf ear, the ganglion cells of the auditory nerve of the deaf ear are triggered, through one or more transfer mechanisms, causing nerve impulses to be sent to the brain through the auditory nerve, which are perceived as sound, and which provide the temporal information that assists the normal-functioning ear.

One such transfer mechanism (by which the electrical stimulus is transferred to the auditory nerve) is through bone conduction. Another transfer mechanism is that the electrical stimulation provided through the mesh ball electrode 10 induces mechanical vibrations in the round window (through tissue contraction) that set up fluid waves and motion within the cochlea (located on the inner-ear side of the round window). The motion of the cochlear fluid caused by these waves tends to bend or move the tiny hair cells located within the cochlea. Movement of the hair cells, in turn, triggers firing of the ganglion cells, causing nerve impulses to be sent to the brain through the auditory nerve which are perceived as sound.

Additionally, it is noted that the present invention may assist in sensing sound by removing or reducing the buzzing or ringing caused by tinnitus, should the person using the invention suffer from tinnitus, which buzzing or ringing interferes with the normal sensing of sound, as described in the pending U.S. patent application Ser. No. 10/932,812, filed Sep. 1, 2004, which application is assigned to the same assignee as is the present application, and is incorporated herein by reference. Further, as previously indicated, the present invention assists in sensing sound by providing temporal information to the deaf ear that supplements the sound signal received by the normal-functioning ear.

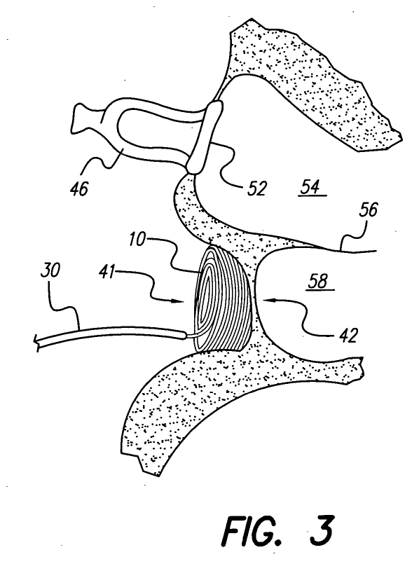

FIG. 3 depicts the middle-ear/inner-ear interface. The oval window 52 separates the scala vestibuli 54 (one of the three parallel ducts that traverses the spiral-shaped cochlea) from the middle ear. The stapes 46 attaches to the oval window 52 on the middle-ear side of the oval window. The stapes 46, in turn, is mechanically coupled through the incus 45 and malleus 44 to the ear drum, or tympanic membrane 47, as seen in FIG. 2. Pressure waves (sound waves) sensed through the outer ear are directed to the tympanic membrane 47 through the ear canal, causing it to vibrate. Such vibrations are then coupled through the malleus 44, incus 45, and stapes 46 of the middle ear to the oval window 52. Vibrations of the oval window in turn cause vibrations of the fluid within the scala vestibuli 54 of the cochlea. Such fluid vibrations are further coupled through the basilar membrane 56 to the scala tympani 58 (another of the parallel ducts that traverse the cochlea). The oval window 52 thus forms a barrier between the scala vestibule 54 and the middle ear; and the round window 42 similarly forms a barrier between the scala tympani 58 and the middle ear. The round window 42 resides in a niche 41, or recess, of the middle ear. This niche 41, or recess, is one preferred extra-cochlear location where the mesh, ball electrode 10 may be placed.

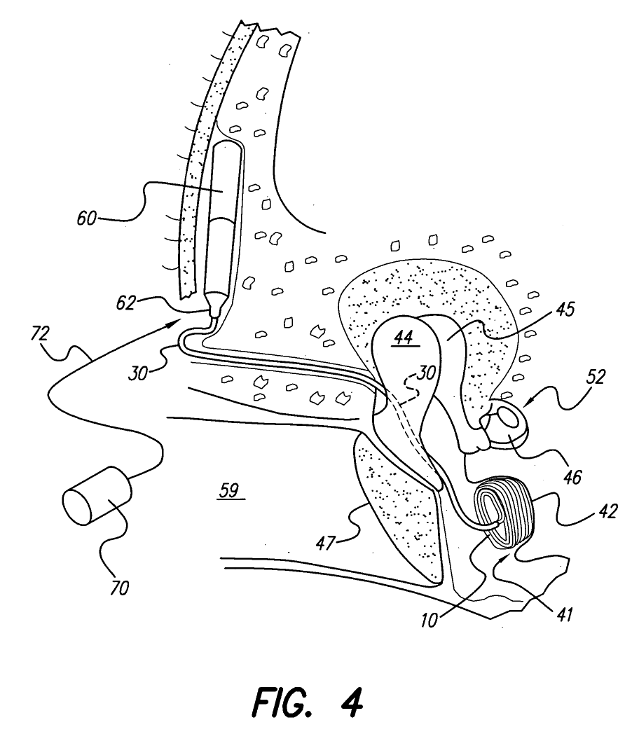

FIG. 4 illustrates a partial side view of outer-ear/middle-ear interface. In a normal-functioning ear, sound waves enter the outer ear through the ear canal 59 and strike the tympanic membrane (ear drum) 47, causing it to vibrate. Such vibrations are transferred through the three tiny bones of the middle ear, the malleus 44, the incas 45, and stapes 46, to the oval window 52. The interface barrier between the outer ear and the middle ear is the tympanic membrane 47. The interface between the middle ear and the inner ear comprises the oval window 52 and the round window 42. As previously indicated, the round window 42 resides within a niche, or recess, 41 of the middle ear. The mesh, ball electrode 10 of the present invention may be placed within the niche or recess 41.

FIG. 4 also shows a preferred placement of an electrical stimulator 60, e.g., a BION® microstimulator device, manufactured by Advanced Bionics Corporation of Valencia, Calif. A BION stimulator 60 is a single channel leadless stimulator, but for purposes of the present invention, may have the cable lead 30 connected thereto by way of a slip-on or snap-on connector 62, or equivalent. The BION stimulator 60 is described more fully, e.g., in U.S. Publication No. US 2004/0059392A1, which publication is assigned to the same assignee as is the present application, and is incorporated herein by reference. A representative connector 62 that may be used to add a lead to such a BION-type stimulator is disclosed in International Publication No. WO 03/063951A1, published Aug. 7, 2003, (International Application No. PCT/US03/02784), also incorporated herein by reference.

As described in the referenced documents, one preferred embodiment of a BION microstimulator includes its own rechargeable power source, i.e., a rechargeable battery. Other BION microstimulators may receive operating power through a close-field RF field. Either type of microstimulator—powered from a self-contained rechargeable power source or from a close-field RF field.—may be used with the invention.

A microphone 70 may be coupled to the stimulator 60 by way of a signal communication link 72. A preferred location for the microphone 70 is in the ear canal of the deaf ear. A preferred link 72 for linking the microphone 70 to the stimulator 60 is a wireless radio frequency (RF) link. However, other suitable links may be used, such as a wire link.

The microphone 70 also preferably includes processing circuitry to process and condition the signal that is sent to the stimulator 60 over the link 72. Such processing circuitry detects the sound or acoustic signals sensed by the microphone's transducer, converts them to electrical signals, amplifies the electrical signals, and processes the amplified electrical signals to determine if they represent an appropriate signal that should trigger the BION stimulator so as to cause it to generate an electrical stimulation pulse that is sent to the mesh, ball electrode 10. Such processing, in one embodiment, involves amplifying and filtering the electrical signal received from the microphone's transducer, and sending a trigger signal to the BION stimulator 60 only when the amplified and filtered signal falls within a prescribed frequency band, e.g., 240-880 Hz, and has an intensity (amplitude) above a prescribed threshold level.

In operation, the mesh, ball electrode 10 is placed in the recess on the middle ear side of the round window 42, or at or in some other suitable extra-cochlear location within the middle ear, of the user's deaf ear. The cable 30 is routed and connected to the stimulator 60. The stimulator 60 is then coupled to the microphone 70, or other external programming device, so as to cause the stimulator 60 to generate appropriate stimuli that provides temporal information to supplement the sound signal received by the other, normally-functioning ear. The stimuli pattern, or regime, will vary from patient to patient, but will typically involve applying mono-polar biphasic stimulus currents at a rate synchronized with the acoustic signals sensed through the microphone 70, at a relatively low current level, e.g., less than 1 or 2 ma peak, applied between the mesh, ball electrode 10 and a suitable return electrode. Typically, the return electrode will be located on the case of the stimulator 60, but it may also be placed in other suitable locations by way of an additional lead or cable connected to the stimulator, or an additional electrode placed on the cable 30 (but having it's own separate electrical connection).

As described above, and in summary, it is thus seen that the present invention involves the use and placement of an extra-cochlear electrode connected to a micro-stimulator, such as a BION microstimulator. Advantageously, the BION microstimulator may be implanted under local anesthesia, thereby significantly reducing the cost and trauma associated with cochlear implant surgery.

In one preferred embodiment, the extra-cochlear electrode may comprise a mesh ball electrode such as that described in pending U.S. patent application Ser. No. 10/932,812, filed Sep. 1, 2004, previously incorporated herein by reference. As disclosed in pending application Ser. No. 10/932,812, one preferred embodiment comprises a mesh ball electrode having a doughnut shape.

The mesh ball electrode 10, or other similar electrode, may be placed in the round window niche, on the promontory, or in some other extra-cochlear location, of the deaf ear in a unilaterally deafened individual.

Connection of the mesh ball electrode is made via a thin wire that is routed, through a standard middle ear surgical procedure that can be performed under local anesthesia, behind the skin and along the bone of the ear canal, to a BION microstimulator, or similar small electrical stimulator.

The microstimulator, or other small stimulator, is placed under the skin or recessed in the temporal bone or some other location in the skull.

In one preferred embodiment, the microstimulator contains its own power supply, e.g., a rechargeable power supply, or may receive operating power from an external power source through close-field RF coupling.

The microstimulator has the ability to generate a stimulation signal derived from the acoustic input collected from the environment. Such stimulation signal is applied through the mesh electrode in order to apply electrical stimulation to the middle ear location where the electrode is positioned.

A microphone, or similar transducer, collects acoustic input from the environment, and is coupled to the microstimulator so that when the acoustic input meets certain prescribed criteria, e.g., exceeds a prescribed intensity threshold, or has frequency components above a certain intensity within a prescribed frequency band, the microstimulator generates a stimulation pulse that is applied to the mesh electrode. The microphone may be worn externally to the stimulator and interface with the stimulator via a wireless radio frequency (RF) link. Alternatively, the microphone may be connected to the simulator via a wired link.

The signal generated by the microstimulator and applied through the mesh ball electrode 10 provides temporal information to supplement the sound signal received by the other, normally functioning ear.

While the invention herein disclosed has been described by means of specific embodiments and applications thereof, numerous modifications and variations could be made thereto by those skilled in the art without departing from the scope of the invention set forth in the claims.

1. A method of providing electrical stimulation to a patient, the method comprising:
(a) identifying the patient has unilateral hearing loss, said patient having a good ear through which sound signals are received and understood and a deaf ear through which sound signals are generally not received and understood;
(b) placing an extra-cochlear electrode in the middle ear portion of the deaf ear;
(c) collecting acoustic signals from the environment surrounding the patient; and
(d) applying electrical stimulation pulses to the extra-cochlear electrode which are derived from the collected acoustic signals,
wherein the electrical stimulation applied to the deaf ear supplements sound signals received by the good ear.

2. The method of claim 1 wherein step (b) comprises inserting a mesh ball electrode near the round window of the patient's deaf ear.

3. The method of claim 2 wherein the mesh ball electrode comprises a ball electrode of diameter 1.5 to 2.5 mm.

4. The method of claim 2 wherein step (b) further comprises implanting a microstimulator near the deaf ear, wherein the microstimulator includes means for generating an electrical stimulation pulse in response to a trigger signal; electrically connecting the mesh ball electrode to the microstimulator; and sending a trigger signal to the microstimulator in response to receiving collected acoustic signals.

5. The method of claim 4 wherein the trigger signal to the microstimulator is sent only when the amplified and filtered signal falls within a prescribed frequency band.

6. The method of claim 5 wherein the prescribed frequency band comprises 240-880 Hz.

7. The method of claim 4 wherein step (c) comprises:
(1) placing a microphone on or in the deaf ear, wherein the microphone includes means for sensing acoustic signals present near or within the deaf ear, means for processing the sensed acoustic signals, and means for generating a trigger signal when the processed acoustic signals meet prescribed criteria; and
(2) coupling the trigger signal generated by the microphone to the implanted microstimulator, whereby an electrical stimulus is generated and applied to the mesh ball electrode whenever the processed acoustic signals meet prescribed criteria.

8. A system for electrically stimulating a patient, the system comprising:
means for identifying the patient has unilateral hearing loss, said patient having a good ear through which sound signals are received and understood and a deaf ear through which sound signals are generally not received and understood;
an extra-cochlear electrode adapted to be positioned in the middle-ear portion of the deaf ear;
a microstimulator adapted to be implanted near the deaf ear, the microstimulator including means for generating a stimulation pulse in response to a trigger signal;
a lead that electrically connects the extra-cochlear electrode with the microstimulator; and
a microphone unit adapted to be placed in or near the deaf ear that includes a transducer for sensing acoustic signals and converting the sensed acoustic signals to electrical signals, means for processing the sensed acoustic signals to determine if they meet prescribed criteria; and means for generating the trigger signal when the prescribed criteria are present in the sensed acoustic signal;
wherein an electrical stimulus is generated by the microstimulator and applied to the extra-cochlear electrode in the middle ear of the deaf ear whenever the sensed acoustic signals meet prescribed criteria, wherein said electrical stimulus is adapted to provide temporal information that supplements acoustic signals received by the good ear.

9. The system of claim 8 wherein the extra-cochlear electrode comprises a mesh ball electrode.

10. The system of claim 9 wherein the mesh ball electrode comprises a ball electrode of diameter 1.5 to 2.5 mm.

11. The system of claim 9 wherein the middle ear includes a round window niche and a promontory, and wherein the mesh ball electrode is adapted to be inserted in the round window niche or on the promontory.

12. The system of claim 9 further including a removable connector that may be detachably secured to the microstimulator, and wherein the lead includes a proximal end and a distal end, and wherein the proximal end of the lead is connected to the removable connector and wherein the mesh ball electrode is located at the distal end.

13. The system of claim 8 wherein the lead comprises a multi-strand insulated cable having a mesh ball electrode of diameter 1.5 to 2.5 mm at a distal end, wherein the mesh ball electrode is adapted to be inserted into the middle ear and wedged into a recess in front of the round window.
